(19) United States
(12) Patent Application Publication
(10) Pub. No.: US 2016/0192937 A1
(43) Pub. Date: Jul. 7, 2016

(54) ADAPTOR WITH FLEXIBLE TIP COUPLED TO A WIRE NEEDLE FOR A CIRCULAR STAPLER OGIVAL TROCAR

(71) Applicants: Antonio Talvane Torres de OLIVEIRA, Barretos/SP (BR); Armando Geraldo Franchini MELANI, Barretos/SP (BR); Croider Franco LACERDA, Barretos/SP (BR); Paulo Anderson BERTULUCCI, Barretos/SP (BR)

(72) Inventors: Antonio Talvane Torres De Oliveira, Barretos/SP (BR); Paulo Anderson Bertulucci, Barretos/SP (BR); Armando Geraldo Franchini Melani, Barretos/SP (BR); Croider Franco Lacerda, Barretos/SP (BR)

(21) Appl. No.: 14/438,389

(22) PCT Filed: Sep. 27, 2013
(86) PCT No.: PCT/BR2013/000374

§ 371 Date: Apr. 24, 2015

(30) Foreign Application Priority Data
Sep. 26, 2013 (BR) 10 2013 024695 6

(51) Int. Cl. A61B 17/115; A61B 17/34; A61B 1/04; A61B 17/04; A61B 17/11; A61B 1/313; A61B 17/06

(52) U.S. Cl. CPC A61B 17/1155; A61B 1/3132; A61B 17/3417; A61B 17/06066; A61B 17/0469; A61B 17/1114; A61B 1/04

(57) ABSTRACT

Flexible tip adapter coupled to a needled wire for a circular stapler ogive trocar invention relates to a surgical instrument that has a flexible tapered end, hollow or solid, continued with surgical thread of varying material and caliber, needled with a straight, curved or semi-curved needle and another rigid end which can be coupled to the rod of the circular surgical stapler. Its use occurs primarily in video laparoscopic surgery, providing a safe transfixion of hollow viscera tissues to be anastomosed. It aims to facilitate the manipulation of the trocar/ogive rod assembly and to minimize the risk of inadvertent injuries to the tissue being anastomosed, consequently reducing the risk of anastomotic fistula.

This patent requirement deals with the invention of a flexible tip adapter coupled to a needled wire for a circular stapler ogive trocar, more specifically of an adapter device with a flexible tip coupled to a needled wire for a circular stapler trocar, used for the penetration and fixation of tissues that will be anastomosed, especially during laparoscopic surgery.

### TECHNICAL BACKGROUND

Currently, circular surgical staplers are generally used to anastomose hollow viscera tissues during digestive tract surgeries, using staples to attach tissues and a cutting blade to create a continuity solution between the structures to be anastomosed. These were initially designed for a use in laparotomy surgery, but lately they have seen an increasing use in video laparoscopic surgeries.

They are comprised of two main parts, the ogive, which is composed by a support plate and a rod, and the effector piece, comprised of an elongated rod where the staples and the blade to be used for the anastomosis of tissues can be found; at one end, there are ogive approaching mechanisms and, at the other end, staple application and sectioning mechanisms.

The ogive can be connected to parts named trocars, which can be used for penetrating tissues to be anastomosed. Trocars for circular stapler ogive rods that can be currently found in the market have a stiff conical tip, which require an adaptation for needled wire so they can be used for penetrating tissues, especially during a video laparoscopic surgery.

This rigid and punctiform end conformation hinders the manipulation of the trocar and ogive assembly, when connected and used in video laparoscopic surgery, creating a risk of injury to tissues adjacent to its location. When used to transfix tissues with the aid of needled wire during video laparoscopic surgeries, the path taken by the trocar may be different from the path that the wire takes, causing damage to the tissues of the organ that is involved and increasing the risks of stapling failure and the occurrence of anastomotic fistula.

### STATE OF THE ART

The current state of the art anticipates only a patent document that, while it may not be considered as having the same focus and purpose, serves to corroborate the fundamentals of the technique here presented, especially regarding the existence of conventional bags, such as P10700984-4, entitled “GASTRIC TROCAR”, comprising a device that anchors itself to the stomach wall and a tube between the device and the patient's oral cavity, including a central axial channel for the passage of laparoscopic instruments, this said device having basically a hollow spool shape provided with a first proximal shield and a second distal shield; the annular channel located between the aforementioned first and second shields has a thickness equivalent to the gastric wall width, in which the device is inserted, with a rigid first proximal shield and with a substantially disk-shaped format in a preferred embodiment; the second shield is rigid and its distal tip has an approximately conical shape, with the smaller base facing forward. The trocar further comprises a sleeve made of impervious material, extending from the mouth of the patient up to the aforementioned tip, which it is fixed to by means of a watertight joint.

In this case, the mentioned patent presents as its inventive concept a device that anchors to the gastric wall for video laparoscopic surgery; however, both its first and second shields are rigid, something that results in the same drawbacks that have been set forth above.

### NEW PRODUCT

Having a knowledge of the state of the art, its gaps and limits, the inventor, after studies and researches, created the flexible tip adapter coupled to a needled wire for a circular stapler ogive trocar, more specifically an adapter device with a flexible tip coupled to a needled wire for a circular stapler trocar, used for the penetration and fixation of tissues that will be anastomosed, especially during laparoscopic surgery.

The flexible tip adapter coupled to a needled wire for a circular stapler ogive trocar proposes a surgical instrument designed to facilitate the manipulation of the trocar/ogive rod assembly and to minimize the risk of inadvertent injuries to the tissue being anastomosed, consequently reducing the risks of anastomotic fistula and inadvertent injuries.

### ADVANTAGES

In short, the claimed adapter has as its most prevalent advantages:
It facilitates the manipulation of the trocar/ogive rod assembly;
It reduces the risk of injuries;
It minimizes the risk of inadvertent injuries in tissues to be anastomized;
It reduces the risk of anastomotic fistula;
It ensures the patient will undergo a safer procedure;
With this reduction in injuries, it accelerates the recovery process of post-surgery patients;
It is easy to handle;
It is built in a simple manner;
It has a great cost x benefit ratio.

### DESCRIPTION OF DRAWINGS

Below, the invention is explained through a reference to the attached graphics, which are shown in an illustrative and non-limiting fashion:

FIG. 1: Side view of the malleable cone that makes up the flexible tip adapter coupled to a needled wire for a circular stapler ogive trocar;

FIG. 2: Perspective view of the flexible cone that makes up the flexible tip adapter coupled to a needled wire for a circular stapler ogive trocar;

FIG. 3: Top view of the flexible cone that makes up this invention;

FIG. 4: Perspective view of the rigid portion of the ogive trocar adapter, in a preferred but not mandatory construction;

FIG. 5: Perspective view of the rigid portion of the adapter in the previous Figure, with internal details in dashed lines;

FIG. 6: Inferior perspective view of the rigid portion of the trocar adapter in FIGS. 4 and 5;

FIG. 7: Perspective view of the trocar adapter in FIGS. 4, 5 and 6, with a particular emphasis on the locking elements;

FIG. 8: Perspective side view of the whole ogive trocar adapter, according to the construction in FIGS. 4 to 7;

FIG. 9: Bottom side perspective view of the ogive trocar adapter, according to the construction in FIGS. 4 to 7, illustrating one of the trocar models that exists in the market and to which it can be fitted;

FIG. 10: Bottom side perspective view of the ogive trocar adapter, connected to one of the trocar models that exists in the market and to which it can be fitted;

FIG. 11: Perspective view showing trocar details in dashed lines, connected to the adapter in FIGS. 9 and 10;

FIG. 12: Side perspective view of the ogive trocar adapter and of another model of trocar that exists in the market and to which it can be fitted;

FIG. 13: Perspective view of trocar models that exists in the market and to which the adapter can be fitted;

FIG. 14: Perspective view showing the adapter connected to the trocar and the circular stapler ogive in one of the possible surgical uses for creating hollow viscera anastomosis;

FIG. 15: Perspective view of the ogive-trocar-adapter assembly, showing a complete resection of the stomach.

FIG. 16: Perspective view showing the introduction of the ogive-trocar-adapter assembly into the esophagus.

FIG. 17: A view of the esophagus closed through suture with needle wire, externalized in its front wall, enabling the ogive-trocar-adapter assembly to be pulled;

FIG. 18: A view showing the emergence of the malleable part of the adapter at the esophagus wall, carrying out a blunt dissection;

FIG. 19: A view of the ogive rod-trocar-adapter assembly externalized at the esophagus wall;

FIG. 20: A view of the trocar-adapter assembly already separated from the ogive rod, with this one already able to receive the stapling device for creating the anastomosis.

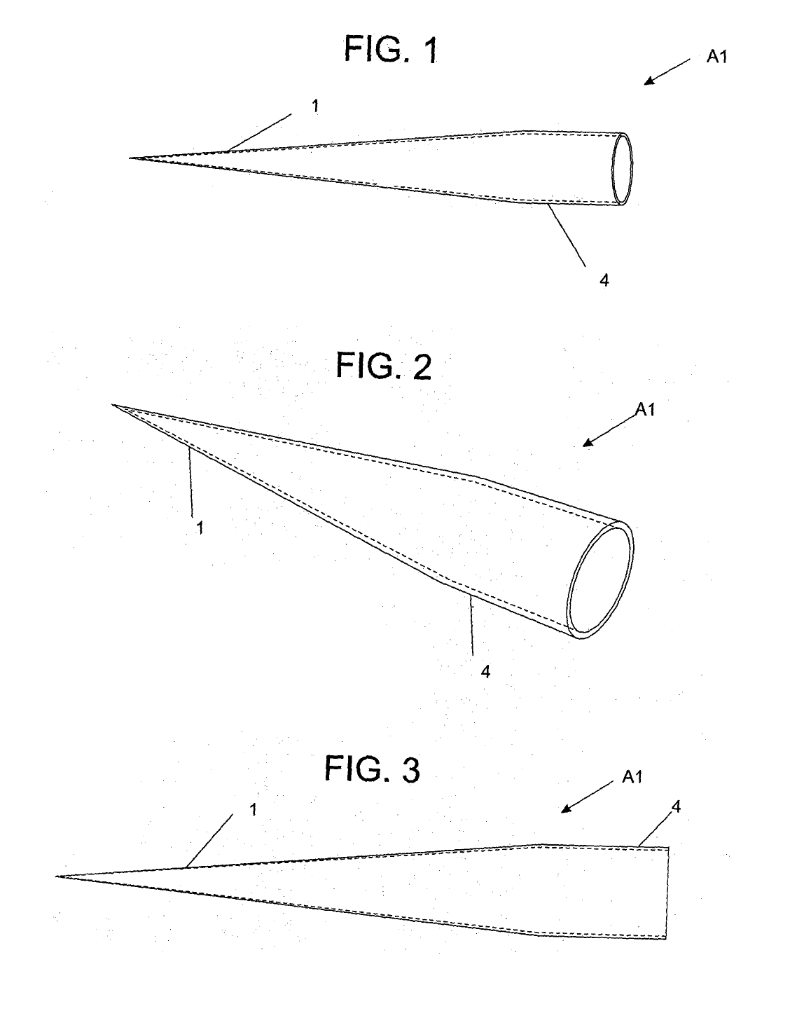

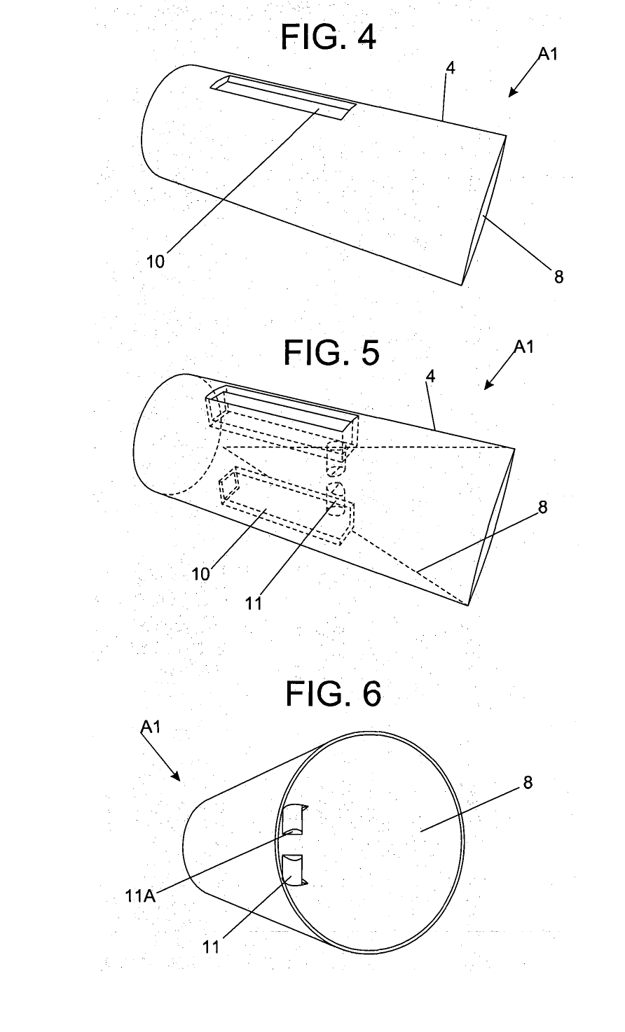

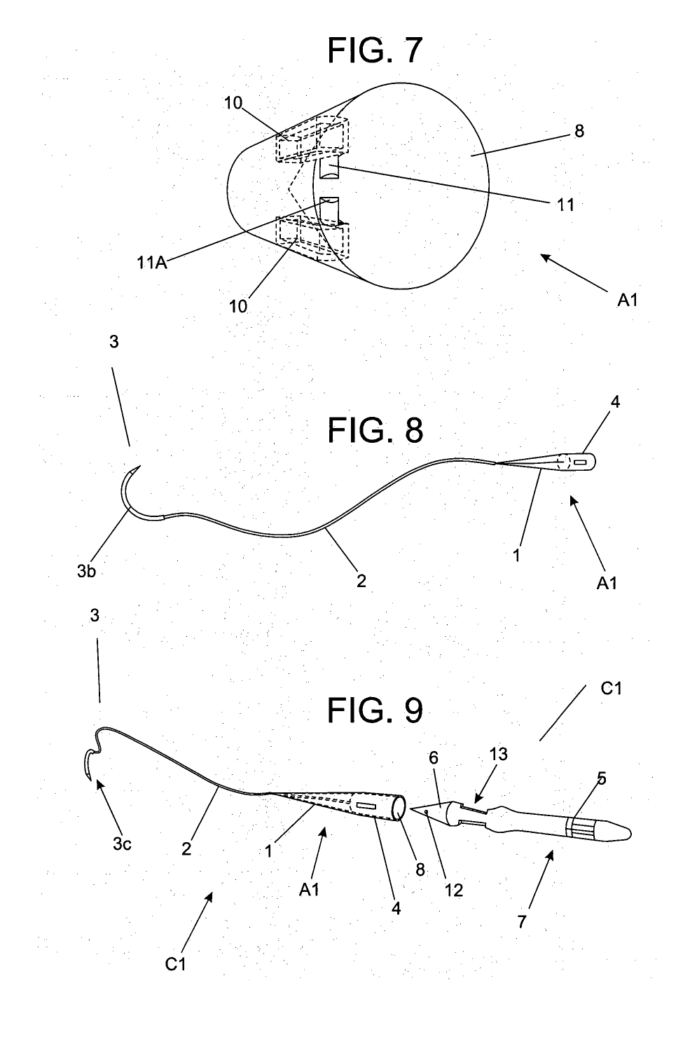

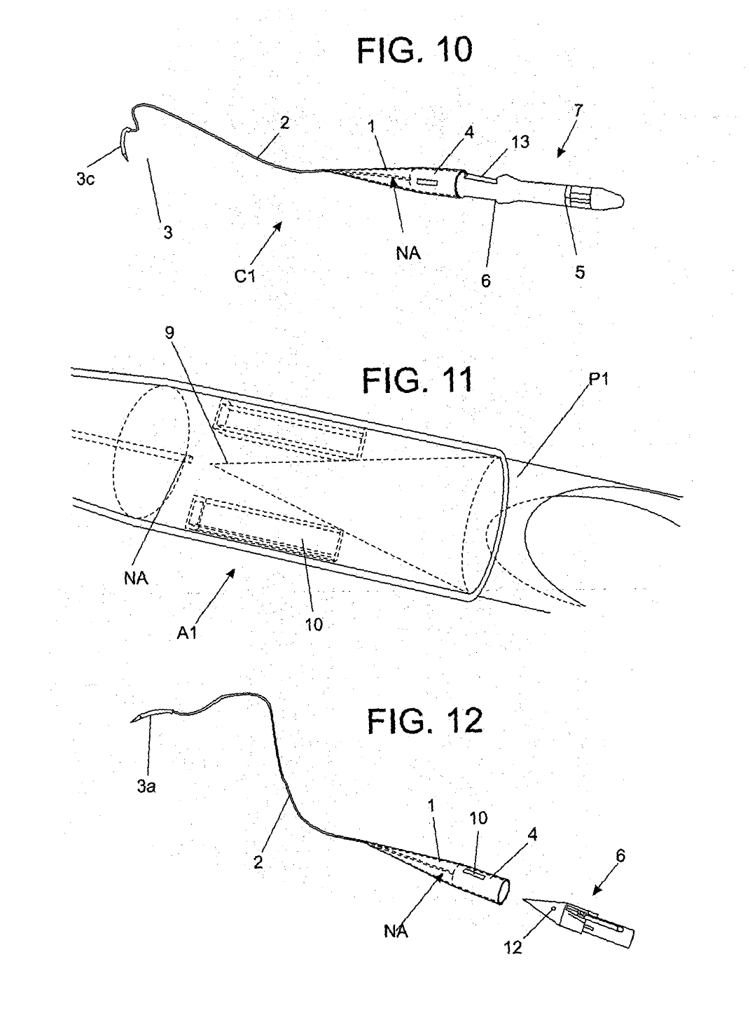

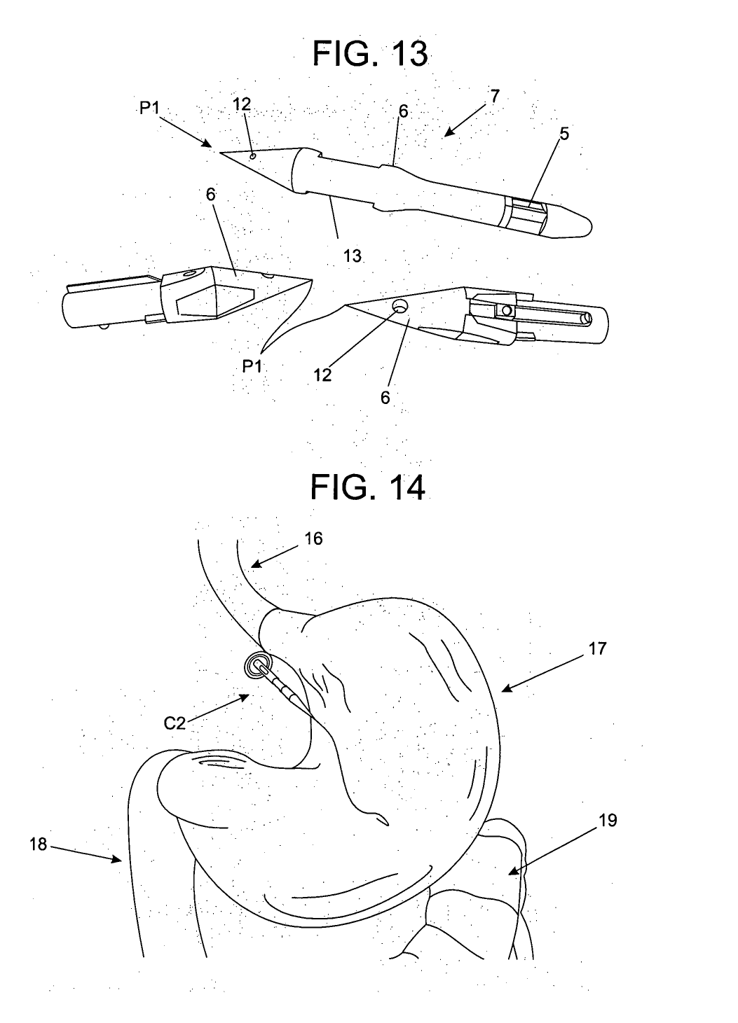

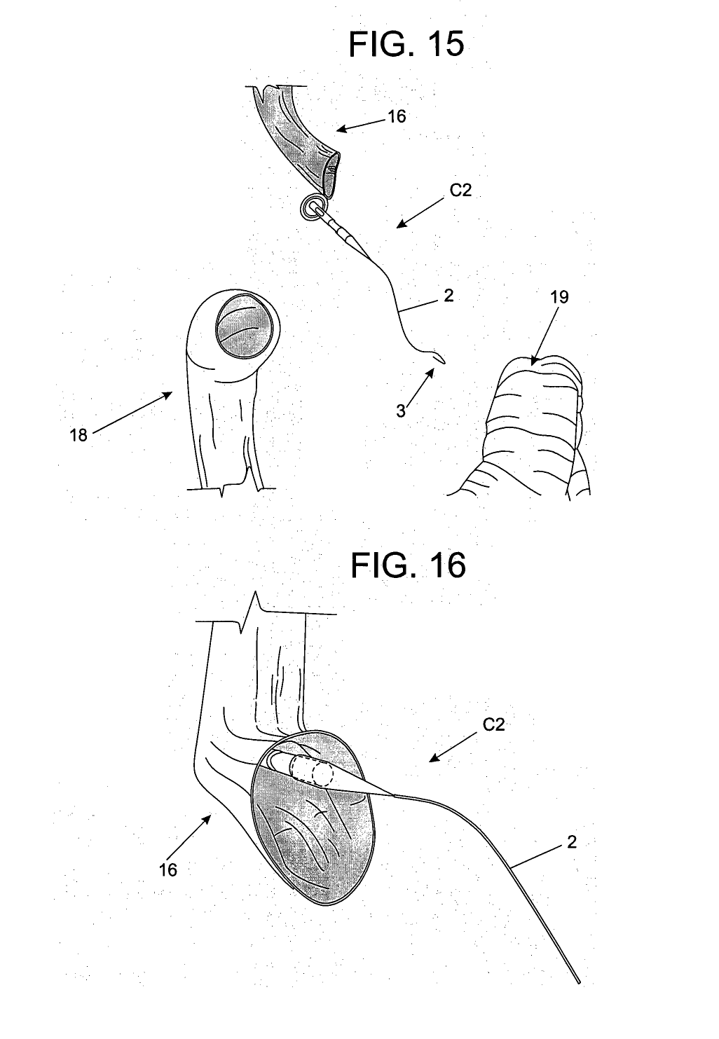

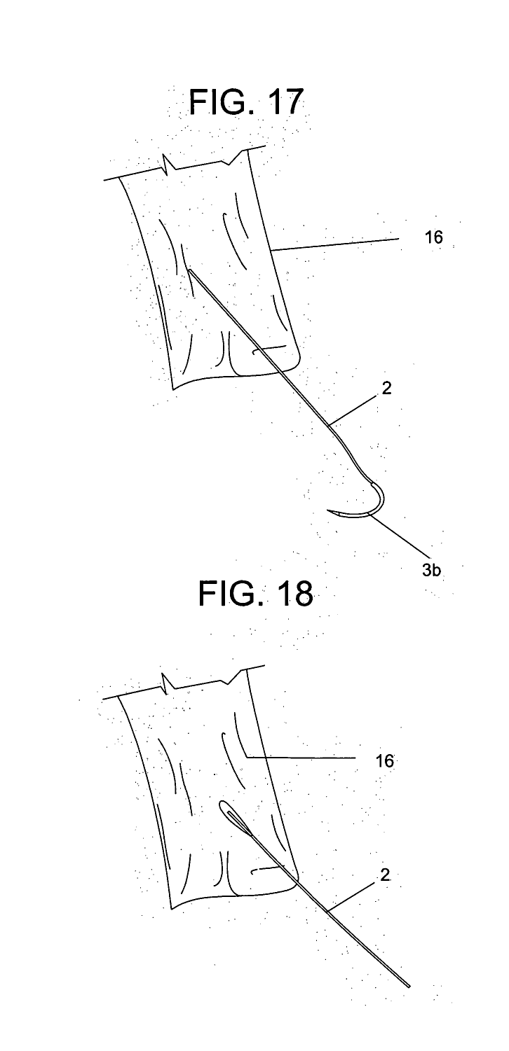

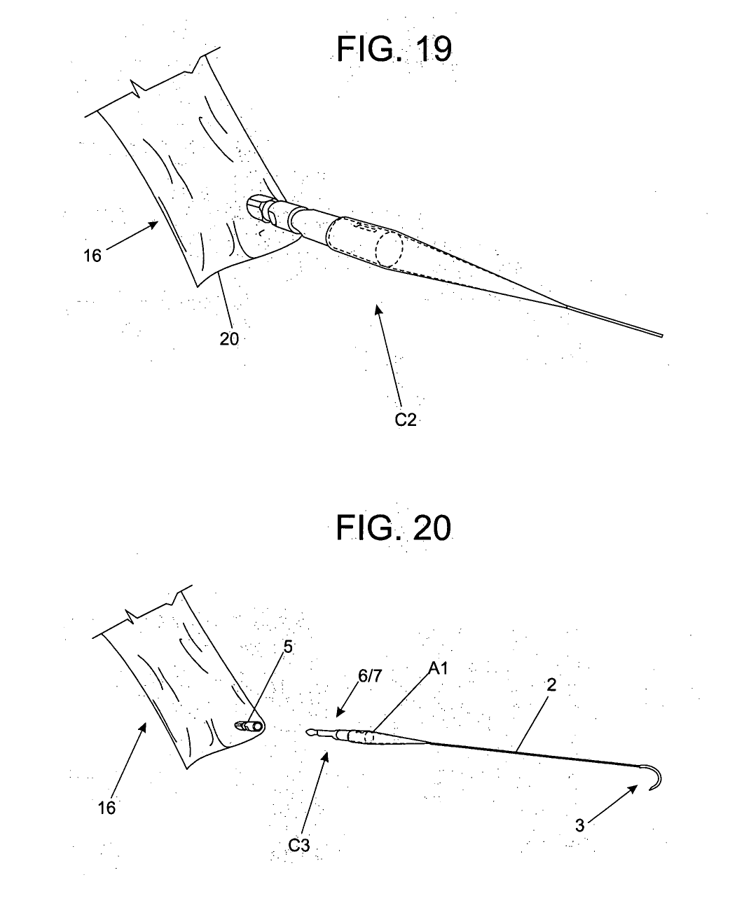

### DETAILED DESCRIPTION

The flexible tip adapter coupled to a needled wire for a circular stapler ogive trocar, which is the object of this invention patent application, refers to a surgical instrument in the form of an adapter (A1) with a flexible tapered end (1), hollow or solid, which continues with surgical thread (2) of variable material and caliber, needled (3) with a straight (3a), curved (3b) or semi-curved needle (3c) and with another rigid end (4) which can be coupled to the tip (P1) of the assembly (C1) comprised by a trocar (6)/ogive rod (7) of a circular surgical stapler. Its use occurs primarily in video laparoscopic surgery, providing a safe transfixion of hollow viscera tissues to be anastomosed. It aims to facilitate the manipulation of the trocar/ogive rod assembly and to minimize the risk of inadvertent injuries to the tissue being anastomosed, consequently reducing the risk of anastomotic fistula.

That is, the adapter (A1) connects, at its tapered end (1), a needle wire (2) with a straight or semicircular cylindrical needle (3), for penetration tissues, the variation of shape and material being according to the choice or the need of the surgeon (usually, a few specific types of surgical steel). An important feature of this tapered end (1) and needle wire (2) assembly is that it allows a greater safety in handling trocar/ogive assembly (C1) due to its flexible tip for penetrating tissues, the edges of which are continued with needle wire (2), which prevents the trocar (6) in taking a different path than the (2) wire, decreasing the risk of inadvertent injury to tissues that are being manipulated.

The adapter (A1) has a rigid (4), massive and cylindrical region, which precedes the conical and tapered end (1). This rigid region (4) contains, in its assembly, an inner tapered portion (8), which originates from the coupling end to the set (C1) formed by the trocar (6)/ogive (7) rod (5) and which protrudes almost through the entire length of the adapter (A1); between the tapered portion (8) and the inner circumference of the said adapter (A1) are bases (10) that receive latches (11) in the form of flexible pins, with these latches having chamfered ends (11A) and being locking cooperative, with respective middle type holes (12) provided in the corresponding end of the trocar (6)/ogive (7) rod (5) assembly (C1), generating a coupling between both parts.

As mentioned previously, the tip (P1) of the trocar (6)/ogive (7) rod (5) assembly receives the rigid end fitting (4) of the adapter (A1), while the tapered end (1) of this adapter is, as mentioned before, malleable and can receive (2) a surgical needle wire (3) that is fixed by its non-needle end (NA) to the central portion of the other end of the tip (P1), which can be cylindrical or not.

The trocar (6) configures slots (13) for the surgeon to insert a plier during the procedure.

In respect to materials, this wire (2) can, optionally, be made of nylon, cotton, silk and polypropylene, among others.

In regards to the tapered end (1), this is made of a malleable material, such as silicone, and can be solid or hollow.

### EXAMPLE OF INVENTION APPLICATION

One of the uses of this adapter is in performing esophageal-jejunal anastomosis. In a total gastrectomy surgery, the esophagus (16), stomach (17), duodenum (18), the colon (19) and the trocar-ogive adapter assembly (C2) are visualized, and this has, increasingly, on a daily basis, been performed through laparoscopy; however, the realization of an esophageal-jejunal anastomosis via laparoscopy can be extremely laborious.

This device plays a crucial part in making such anastomosis easier. The surgeon, after performing a gastric resection, inserts the trocar-ogive adapter assembly (C2) inside the esophagus (16) and, using the laparoscopic needle carrier, used the wire (2) needle (3) from the adapter to transfix the anterior esophagus wall (16) about an inch above its section line, externalizing the needle (3) and part of the wire (2). Then, the surgeon carries out the closure (20) of the esophagus (16), which can be accomplished with the laparoscopic linear stapler cutter or through manual suturing.

After this has been done, with the aid of a grasping plier, the surgeon starts to pull the wire (2) from the device, causing the malleable part (1) to appear through a blunt dissection; he or she keeps on pulling until the rod (5) of the ogive (7) is sufficiently visible, thus eliminating the need for carrying out a purse string suture to lock the ogive (7). Once it is well positioned, the adapter-trocar assembly (C3) can de unattached from the ogive (7) rod (5), allowing it to receive the circular stapling device, which will be introduced in the jejunal loop so the anastomosis can be carried out.

Therefore, the invention relates to a surgical instrument that has a flexible tapered end, hollow or solid, continued with surgical thread of varying material and caliber, needled with a straight, curved or semi-curved needle and another rigid end which can be coupled to the rod of the circular surgical stapler. Its use occurs primarily in video laparoscopic surgery, providing a safe transfixion of hollow viscera tissues to be anastomosed. It aims to facilitate the manipulation of the trocar/ogive rod assembly and to minimize the risk of inadvertent injuries to the tissue being anastomosed, consequently reducing the risk of anastomotic fistula.

Finally, this application for an invention patent has a novelty and inventive activity by being able to ensure a better handling of the trocar/ogive rod assembly, reducing the risk of injuries and minimizing the risk of inadvertent injury to the tissues being anastomosed, which, added to its industrial application, is worthy of a patent privilege.

What claimed is:

1) Flexible tip adapter coupled to a needled wire for a circular stapler ogive trocar, for use in video laparoscopic surgery, wherein presents an adapter (A1) with a flexible, hollowed or solid tapered end (1), continued with surgical thread (2) of varying material and caliber, needled (3) and another rigid end (4) which can be coupled to the tip (P1) of a trocar (6)/ogive (7) rod (5) assembly (C1) of a circular surgical stapler.

2) Flexible tip adapter coupled to a needled wire for a circular stapler ogive trocar, according to claim 1, wherein the needled (3) is with a straight (3a), curved (3b) or semi-curved (3c) needle.

3) Flexible tip adapter coupled to a needled wire for a circular stapler ogive trocar, according to claim 1, wherein trocar/ogive assembly (C1) has a flexible tip for penetrating tissues, the edges of which are continued with needle wire (2), which prevents the trocar (6) in taking a different path than the (2) wire.

4) Flexible tip adapter coupled to a needled wire for a circular stapler ogive trocar, according to claim 1, wherein the rigid (4), massive and cylindrical region contains an inner tapered portion (8), which originates from the coupling end to the assembly (C1) formed by the trocar (6)/ogive (7) rod (5) and which protrudes almost through the entire length of the adapter (A1).

5) Flexible tip adapter coupled to a needled wire for a circular stapler ogive trocar, according to claim 1, wherein , in a preferred construction, between the tapered portion (8) and the inner circumference of the said adapter (A1), it presents bases (10) that receive latches (11) in the form of flexible pins, with these latches having chamfered ends (11A) and being locking cooperative, with respective middle type holes (12) provided in the corresponding end of the trocar (6)/ogive (7) rod (5) assembly (C1), generating a coupling between both parts.

6) Flexible tip adapter coupled to a needled wire for a circular stapler ogive trocar, according to claim 1, wherein the tip (P1) of the trocar (6)/ogive (7) rod (5) assembly receives the rigid end fitting (4) of the adapter (A1), while the tapered end (1) of this adapter is malleable and can receive (2) a surgical needle wire (3) that is fixed by its non-needle end (NA) to the central portion of the other end of the tip (P1), which can be cylindrical or not.

7) Flexible tip adapter coupled to a needled wire for a circular stapler ogive trocar, according to claim 1, wherein the trocar (6) configures slots (13) for inserting a plier.

8) Flexible tip adapter coupled to a needled wire for a circular stapler ogive trocar, according to claim 1, wherein its thread (2) is, optionally, made of nylon, cotton, silk or polypropylene.

9) Flexible tip adapter coupled to a needled wire for a circular stapler ogive trocar, according to claim 1, wherein its tapered end (1) is made of a malleable material, such as silicone, and which can be solid or hollow.
